(19) United States
(12) Patent Application Publication
(10) Pub. No.: US 2023/0133636 A1
(43) Pub. Date: May 4, 2023

(54) METHODS AND SYSTEMS FOR ACHIEVING EFFICIENT ELECTROCHEMICAL REACTIONS USING A TWO-ELECTRODE SYSTEM

(71) Applicant: TWENTY TWENTY THERAPEUTICS LLC, South San Francisco, CA (US)

(72) Inventors: Zidong Li, South San Francisco, CA (US); Christian Gutierrez, Pacifica, CA (US)

(21) Appl. No.: 17/971,717

(22) Filed: Oct. 24, 2022

Related U.S. Application Data
(60) Provisional application No. 63/272,728, filed on Oct. 28, 2021.

(51) Int. Cl. A61F 9/00; C25F 3/02

(52) U.S. Cl. CPC A61F 9/0017; C25F 3/02

(57) ABSTRACT

Efficient electrochemical reactions can be achieved using a two-electrode system embedded within a silicone hydrogel by using a one-step linear sweep-hold method. A voltage value is initially set to be a voltage that corresponds to a peak current (Vp). An amperometric process is performed at the voltage value. During the amperometric process, the voltage value is corrected as a current shifts.

### RELATED APPLICATIONS

This application claims priority to U.S. Provisional Application Ser. No. 63/272,728, filed Oct. 28, 2021, entitled “METHODS AND SYSTEMS FOR ACHIEVING EFFICIENT ELECTROCHEMICAL REACTIONS USING A TWO-ELECTRODE SYSTEM”. The entirety of this provisional application is hereby incorporated by reference for all purposes.

### TECHNICAL FIELD

The present disclosure relates to electrochemical reactions, and, more specifically, to methods and systems for achieving efficient electrochemical reactions performed within a silicone hydrogel.

### BACKGROUND

Traditionally, an efficient electrochemical reaction can be achieved during an amperometric process using a two-step method (shown in FIG. 1) that includes a first step (STEP 1) of finding a voltage where a peak current occurs (Vp) using linear sweep voltammetry and a second step (STEP 2) of performing electrochemical methods, like amperometry, at Vp. While the traditional two-step method tends to work well when the amperometric process is performed in solution, the two-step method becomes ineffective when the amperometric process is performed within a silicone hydrogel. In order for the amperometry to be at its most effective, the amperometry must be executed immediately when the peak current occurs. However, when the amperometric process is performed within the silicone hydrogel, there is a delay that causes a low actuation current (shown in the current vs. time illustration of STEP 2), which leads to the amperometry being ineffective. Accordingly, the traditional two-step method cannot achieve high efficiency when the amperometric process is performed within the silicone hydrogel.

### SUMMARY

Provided herein are systems and methods that can achieve efficient electrochemical reactions during an amperometric process performed within a silicone hydrogel (the systems and methods can be used to the same benefit with a three-electrode system, but are especially useful with a two-electrode system that is embedded within a silicone hydrogel). The systems and methods can employ a one-step linear sweep-hold method (shown in FIG. 2) to achieve robust electrochemical reactions.

In one aspect, the present disclosure includes a method for achieving efficient electrochemical reactions using a two-electrode system. The method includes setting a voltage value to be a voltage that corresponds to a peak current (Vp); and performing an amperometric process at the voltage value. The voltage value is corrected during the amperometric process as a current shifts.

In another aspect, the present disclosure includes a method for achieving efficient electrodissolution using a two-electrode system. The method includes setting a voltage value to a voltage that corresponds to a peak current (Vp) and applying an electrodissolution process with the voltage set at Vp to actuate opening of a reservoir covered by an electrode. The reservoir can be positioned within a hydrogel of an ophthalmic device. The voltage value is corrected during the electrodissolution process as a current shifts as the electrode is dissolved. The method further includes releasing a therapeutic from the reservoir.

In a further aspect, the present disclosure includes a system that can achieve efficient electrodissolution using a two-electrode system. The system includes a controller that includes a memory storing instructions and a processor configured to access the memory and execute the instructions to set a voltage value that corresponds to a peak current (Vp). The system also includes a generator coupled to the controller to generate an electrical signal at the voltage value and send the electrical signal to an electrode to perform an amperometric process at a peak current. The controller corrects the voltage value as the peak current shifts during the amperometric process.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 shows a traditional two-step method to achieve efficient electrochemical reactions and its limitations regarding activation current when the electrochemical reactions are performed within a silicone hydrogel;

FIG. 2 shows a one-step linear sweep-hold method to achieve efficient electrochemical reactions as an alternative to the traditional two-step method of FIG. 1;

FIG. 3 shows a system that can employ the one-step linear sweep-hold method of FIG. 2 to achieve efficient electrochemical reactions in an amperometric process;

FIG. 4 shows a diagram of an implementation of the system of FIG. 3 with an ophthalmic device undergoing the amperometric process of electrodissolution of a metal film electrode to allow a therapeutic to escape a reservoir and enter a hydrogel matrix;

FIG. 5 shows actions that can be employed by the controller of FIG. 3 to implement the one-step linear sweep-hold method;

FIG. 6 shows an example control diagram for action 1 of FIG. 5, setting the initial voltage;

FIG. 7 shows an example control diagram for action 2 of FIG. 5, adjusting the voltage;

FIG. 8 shows an example control diagram for action 3 of FIG. 5, stop delivering the voltage;

FIG. 9 shows an example process flow diagram of a method for executing the one-step linear sweep-hold method to achieve an efficient amperometric process;

and

FIG. 10 shows an example process flow diagram of a method for more efficient therapeutic release using the one-step linear sweep-hold method to achieve an efficient amperometric process.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “ophthalmic device” refers to a medical instrument used on or within a portion of a patient's eye for optometry or ophthalmology purposes (e.g., for diagnosis, surgery, vision correction, or the like). An ophthalmic device can be “smart” when it includes one or more components that facilitate one or more active processes for purposes other than traditional lens-based vision correction (e.g., therapeutic release).

As used herein, the term “reservoir” refers to a storehouse for a therapeutic with a portion being open for release of the therapeutic from a reservoir (allowing for diffusion of the therapeutic out of the reservoir and into the surrounding hydrogel matrix). The opening may be covered to prevent release of the therapeutic. In some instances, the covering can facilitate release of the therapeutic from the reservoir. For example, at least a portion of the covering can be an electrode that can electrodissolve to facilitate the release of the therapeutic.

As used herein, the term “therapeutic” refers to one or more substance (e.g., liquid, solid, or gas) related to the treatment, symptom relief, or palliative care of a disease, injury, or other malady. The therapeutic can be a pharmaceutical, for example.

As used herein, the term “electrode” refers to a conductive solid (e.g., including one or more metals, one or more polymers, or the like) that receives/transmits an electrical signal. Unless otherwise noted, the term “metal electrode” is used to refer to the “working electrode” of an electrochemical system, which includes the working electrode, a counter electrode, and a reference electrode.

As used herein, the term “working electrode” refers to an electrode (e.g., a metal electrode) on which a reaction of interest (e.g., electrodissolution) is occurring. A non-limiting example of the working electrode is a thin-film gold electrode.

As used herein, the term “reference electrode” refers to an electrode that has a constant electrochemical potential as long as no current flows through it.

As used herein, the term “counter electrode” refers to an electrode that completes the circuit and applies input potential.

As used herein, the term “counter/reference (or vice versa, reference/counter) electrode”, which can also be referred to as a common electrode, refers to a single electrode that performs both the functions of a counter electrode and a reference electrode.

As used herein, the term “two-electrode system” refers to an electrochemical system including a working electrode and a counter/reference electrode.

As used herein, the term “electrical signal” refers to a waveform generated by an electronic means, such as a generator. An electrical signal may be a voltage signal or a current signal.

As used herein, the term “voltage” refers to a potential difference in charge between two points.

As used herein, the term “current” refers to a flow of electrical charge carriers.

As used herein, the term “amperometry” refers to an electrochemical technique in which a constant voltage is set, and can be corrected, to be applied to a working electrode to generate a current based on an electrochemical reaction (or “amperometric process”), such as electrodissolution of a metal electrode.

As used herein, the term “electrodissolution” refers to a process for dissolving a solute using an electrical catalyst. In one non-limiting example, application of an electrical signal to a solid metal can cause the solid metal to dissolve into separate molecules.

As used herein, the term “peak current (Iₚ)” refers to the maximum amount of current that can be generated at a constant voltage based on an electrochemical reaction.

As used herein, the term “instantaneous current (Iₜ)” refers to the current generated at the constant voltage based on the electrochemical reaction at an instance in time.

As used herein, the term “current drop percentage” (ΔI %) refers to a ratio when the instantaneous current at a time (Iₜ) divided by the current at the start of the voltage hold (Iₒ) and the ratio multiplied by 100%.

As used herein, the term “threshold” refers to a value beyond which a certain reaction, phenomenon, result, or condition occurs. For non-limiting example, the threshold for determining when the current drop percentage indicates the electrodissolution processes has been successful can be predefined as any value (e.g., between 1% and 50%.)

### II. Overview

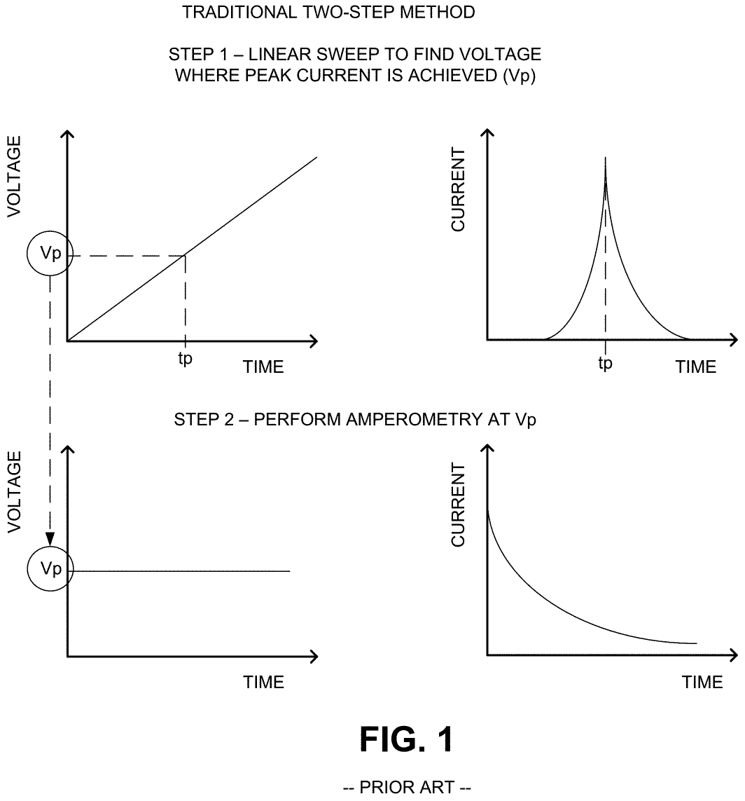

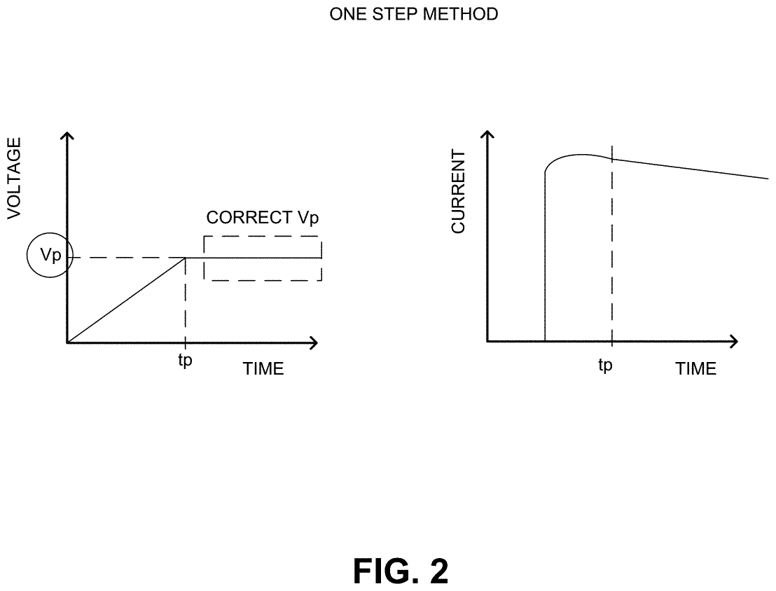

Described herein are systems and methods that can achieve efficient electrochemical reactions during an amperometric process using electrodes embedded within a silicone hydrogel. Traditionally, an efficient electrochemical reaction can be achieved in solution using a two-step method (shown in FIG. 1) that includes a first step (STEP 1) of finding a voltage where a peak current occurs (Vp) using linear sweep voltammetry and a second step (STEP 2) of performing electrochemical methods, like amperometry, at Vp. But the traditional two-step method is quite inefficient when electrodes are embedded within a silicone hydrogel at least because of the rapid current drop that is only magnified when the voltage hold cannot be done immediately after Vp is determined. Accordingly, described herein is a one-step linear sweep-hold method (shown in FIG. 2) that can be employed by the systems and methods described herein to provide an auto programmed all-in-one voltage stimulus step in a closed-loop fashion to achieve robust electrochemical reactions.

This one-step linear sweep-hold method is important when considering its use in a smart ophthalmic device. Smart ophthalmic devices are often constructed of a silicone-hydrogel material that encapsulates components of the device. For example, the device can include a reservoir that can store a therapeutic. To prevent escape of the therapeutic from the reservoir, the reservoir can be covered by a metal film. In order for the therapeutic to be released from the reservoir in a controlled, on-demand, manner, the metal film can undergo the electrochemical process of electrodissolution with the metal film acting as the working electrode. Since the silicone-hydrogel material of the smart ophthalmic device is generally of a small, fixed size, a two-electrode electrochemical system is generally preferred. The two-electrode system has a single reference/counter electrode and the working electrode and has a simpler integration within the silicone-hydrogel than a traditional three-electrode system. However, the two-electrode system embedded within the silicone hydrogel suffers from signal drift of an unreferenced counter electrode and limited ion diffusion within the silicone hydrogel, which each can lead to inefficient electrochemical reactions. The one-step linear sweep-hold method is a significant key to enabling on demand, electronic therapeutic delivery from smart ophthalmic devices that employ two-electrode systems embedded in a silicone hydrogel.

### III. System

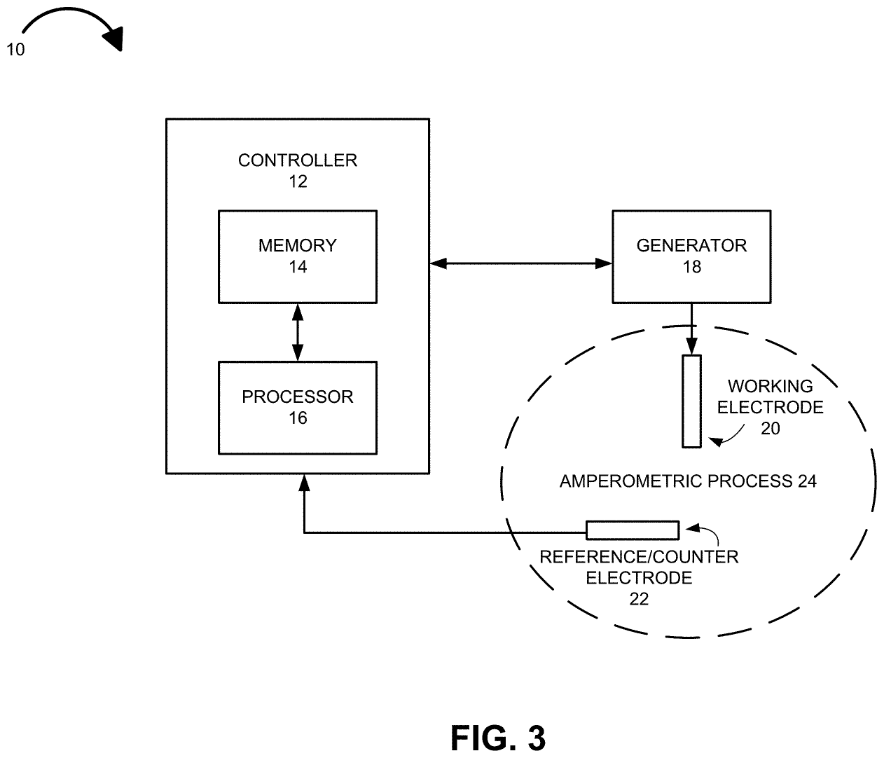

Provided herein is a system 10 (FIG. 3) that can achieve efficient electrochemical reactions of amperometry within a silicone hydrogel. The electrochemical reactions will be described using a two-electrode system, but these electrochemical reactions can also use a traditional three-electrode systems. The efficient electrochemical reactions can be achieved by using the linear sweep-hold method. As illustrated in FIG. 2, the linear sweep-hold method can provide a closed-loop voltage control system for efficient two-electrode amperometry. By using the linear sweep-hold method, robust electrochemical reactions (e.g., electrodissolution) can be achieved in diffusion-limited systems such as silicone hydrogels.

FIG. 3 shows a system 10 that can employ the one-step sweep-hold method of FIG. 2 to achieve efficient electrochemical reactions facilitated by an amperometric process 24. The system includes a controller 12 that is electronically coupled to a generator 18. The electronic coupling can be via a wired connection, a wireless connection, or a connection that is some combination of wired and wireless connection. The controller 12 can provide parameters for an electrical signal to the generator 18. The parameters are determined by the controller 12 based on the one-step linear sweep-hold method. The generator 18 generates an electrical signal according to the parameters.

The controller 12 can store instructions (e.g., computer executable instructions) related to the one-step linear sweep-hold method in a memory 14. The controller 12 can also store additional instructions, data, and information. For example, the controller 12 can be a personal computer, a laptop computer, a workstation, a computer system, an appliance, an application-specific integrated circuit (ASIC), a server, a server BladeCenter, a server farm, etc. The controller 12 can include at least a system bus, a communication link, a processor (or processing unit) 16, and a memory 14, that can be one or more non-transitory memory devices implementing at least a system memory (including a computer readable medium, a memory card, a disk drive, a compact disk (CD), a flash drive, a hard disk drive, server, standalone database, or other non-volatile memory). The processor 16 can be, for example, embedded within one or more application specific integrated circuits (ASICs), digital signal processors (DSPs), digital signal processing devices (DSPDs), programmable logic devices (PLDs), field programmable gate arrays (FPGAs), microprocessors, other electronic units designed to perform the functions of a processor, or the like. The system bus can connect the memory 14 and the processor 16 (e.g., when the memory 14 and processor 16 are separate devices within the controller 12). However, in some instances the memory 14 and the processor 16 can be embodied within the same device (e.g., a microcontroller device). Optionally, the controller 12 can include a display (e.g., a video screen) and/or an input device (e.g., a keyboard, touch screen, and/or a mouse).

As noted, the memory 14 can store instructions related to the one-step linear sweep-hold method. At least a portion of the instructions can be accessed by the processor 16 for execution of at least the portion of the one-step linear sweep-hold method. In its simplest form, as a first step, the processor 16 can access the memory 14 to execute the instructions to set parameter of the electrical signal (e.g., a voltage value, a timing parameter, a desired current to be produced, or the like). For example, the instructions can be to set a voltage value to be a voltage value that corresponds to a peak current (Vp). This parameter is sent to the generator 18, which can generate the electrical signal at the voltage value and send the electrical signal to an electrode (working electrode 20) to perform an amperometric process 24 at a peak current (initially set to Vp). The reference/counter electrode 22 (the second electrode of a two-electrode system) can finish the circuit by sending a return current back to the controller 12. Based on the return current, the controller 12 can detect a shift in the peak current away from Vp. When there is a shift in the peak current, the controller 12 can set the parameter that is sent to the generator 18 to correct the voltage based on any shift in the current that is detected.

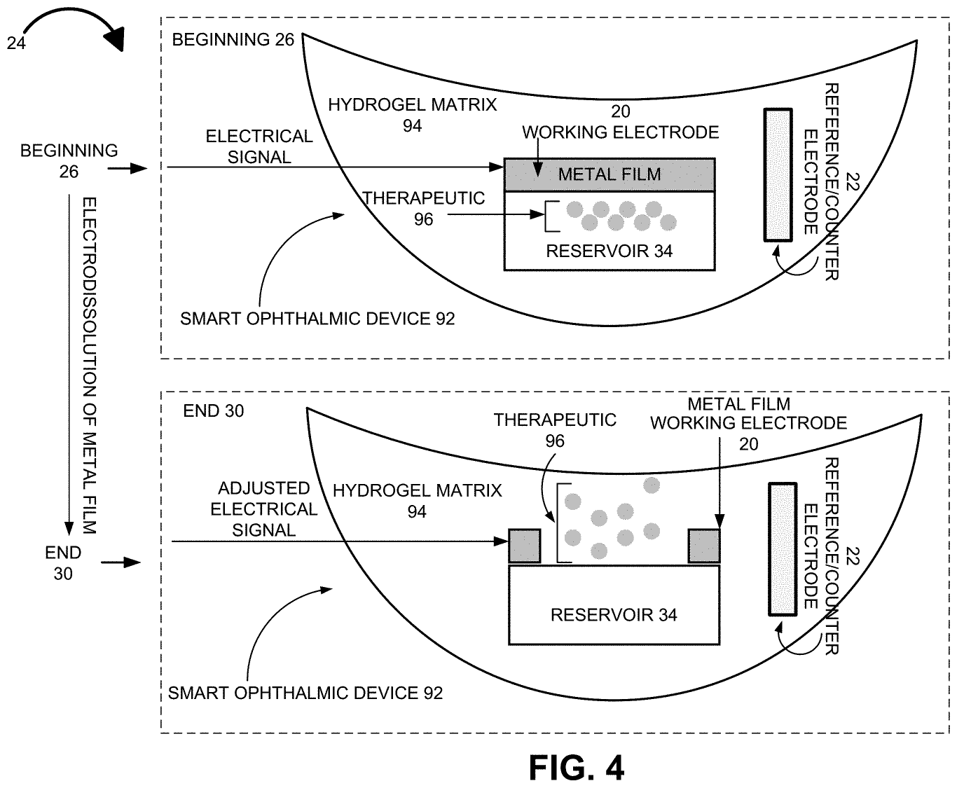

The electrical signal delivered by the generator can be varied based on the parameter received from the controller 12 to increase the efficiency of the amperometric process 24. As shown in FIG. 2, right side, the current remains high after the Vp is set (this is in contrast to the right side of STEP 2 of the two-step process of FIG. 1). The high current facilitates one or more electrochemical reactions of the amperometric process 24. It should be noted that the amperometric process 24 can be any kind of amperometric process, exemplified (but not limited) by an electrodissolution process shown in FIG. 4.

FIG. 4 shows an example amperometric process 24, electrodissolution of a metal film occurring within a hydrogel (e.g., silicone-hydrogel) matrix 94 of a smart ophthalmic device 92. Since the hydrogel matrix 94 of the smart ophthalmic device is of a pre-defined, small size, a two-electrode system (of a working electrode 20 and a reference/counter electrode 22) is preferable (and easier to place within the silicone-hydrogel matrix) than the traditional three-electrode system (with a working electrode, a reference electrode, and a counter electrode). The two-electrode system coupled with a generator (e.g., generator 18 of FIG. 1) can provide the amperometric process, but the amperometric process operates with a large increase in efficiency (e.g., as much as 100× greater efficiency) when the linear sweep-hold method (shown in FIG. 2) is used.

It should be noted that the working electrode 20 doubles as a metal material (e.g., a metal film) covering a reservoir 34 that holds a therapeutic 96 (where the presence of the metal material stops the escape of the therapeutic from the opening in the reservoir). When the metal material of the metal film undergoes electrodissolution the metal material dissolves and the therapeutic 96 can leave the reservoir 34. The thickness, material, and the like, of the metal material can be chosen based on one or more desired properties of the electrodissolution. As an example, shown in FIG. 4, the metal material can be a metal film, which can be a thin gold film (e.g., 0.1 to 1000 nanometers thick) (but the metal film is not so limited).

The beginning 26 and the end 30 of the electrodissolution of the metal film (which also operates as the working electrode 20) are illustrated in FIG. 4. At the beginning 26 of the electrodissolution of the metal film, the metal film (operating as the working electrode 20) can receive an initial electrical signal (from generator 18, not illustrated) set with a voltage value to be a voltage value that corresponds to a peak current (Vp). As the electrodissolution progresses, the reference/counter electrode 22 can return current (to controller 12, not illustrated in FIG. 4) and the controller 12 can determine when a current shift occurs and can determine a new voltage value to be set for the amperometric process. As the metal film undergoes the electrodissolution process, progressively less of the metal film remains to operate as the working electrode 20, so the current will shift. Additionally, the controller 12 can further determine when to end the electrodissolution process (e.g., when the metal film/working electrode 20 has dissolved to a level where the therapeutic 96 is no longer held within the reservoir 34). This is illustrated as the end 30 of the electrodissolution process (when a sufficient amount of the therapeutic 96 is allowed to diffuse out from the reservoir 34).

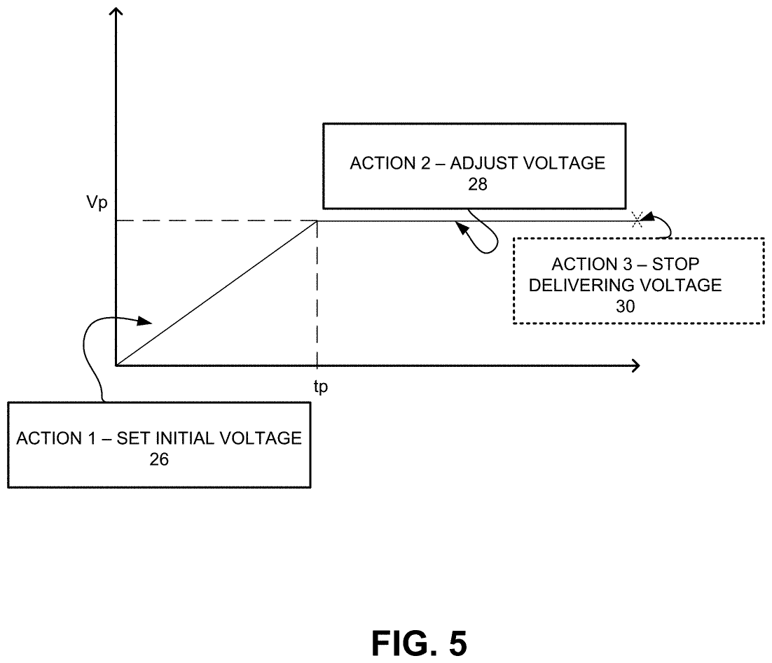

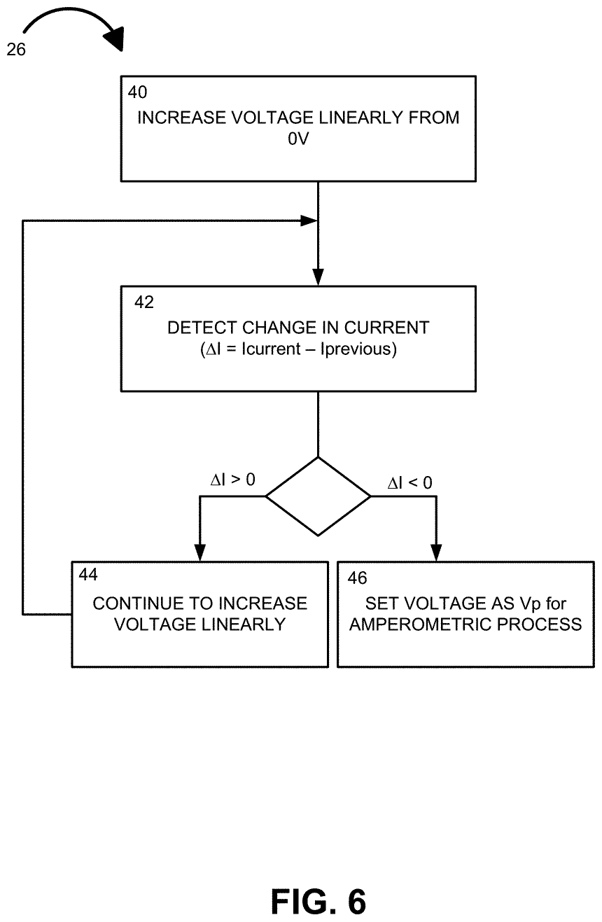

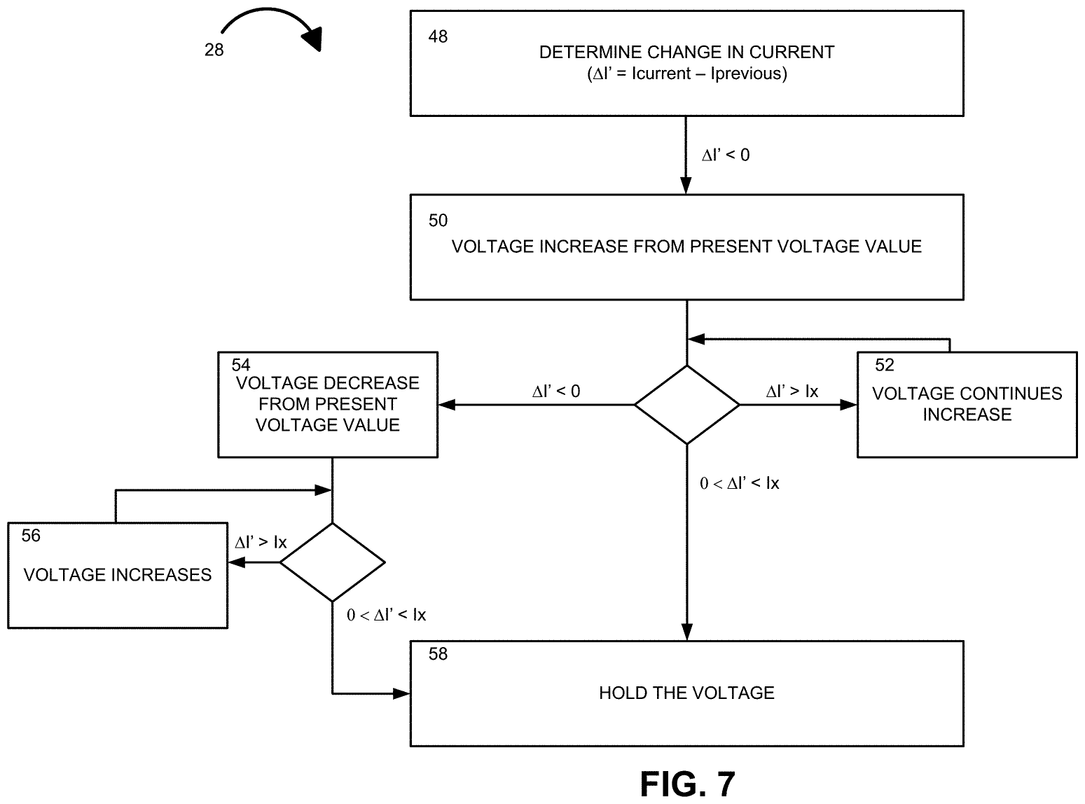

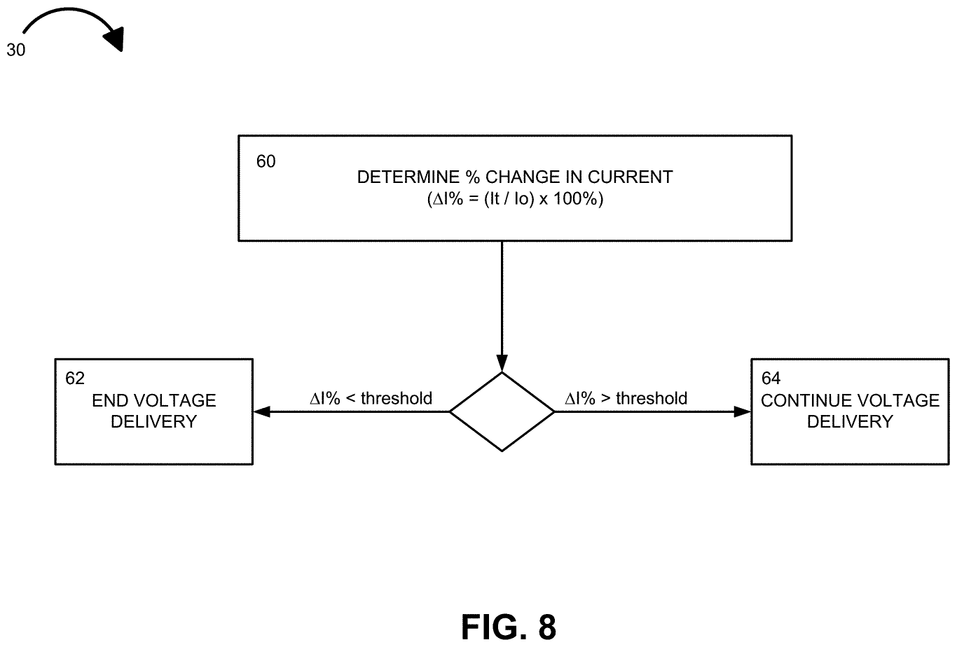

The controller 12 can store different actions in its memory 14 for execution by the processor 16. As shown in FIG. 5, the actions can include “action 1—set initial voltage 26” (shown in more detail in FIG. 6), “action 2—adjust voltage 28” (shown in more detail in FIG. 7), and, optionally, “action 3—stop delivering voltage 30” (shown in more detail in FIG. 8). The actions can be executed together as a common program to form the linear sweep-hold method, in which at least a voltage where a peak current is achieved (Vp) is found and set as an initial voltage value (action 1—set initial voltage 26), then immediately held with subsequent correction of the voltage value as the current shifts as an amperometric process is performed (action 2—adjust voltage 28) and, optionally, stop delivering voltage (action 3—stop delivering voltage 30) to end the amperometric process 24. It should be noted that one or more of action 1, action 2, and action 3 can be performed automatically (e.g., based on execution of one or more programs stored in the memory 14 of the controller 12 by processor 16)

Referring now to FIG. 6, action 1—set initial voltage 26 is shown in greater detail. At 40, the action starts with the voltage at 0 V and increases the voltage linearly to 0 V+δV (a predefined step). At each voltage step, the current I caused by the voltage is detected. At 42, a change in current (ΔI=Icurrent−Iprevious) is detected with each voltage increase. Icurrent is the current measured by the counter/reference electrode 22 at the present time and Iprevious is the current measured by the counter/reference electrode the previous measurement time. At 46, if ΔI is less than zero (Iprevious>Icurrent) or equal to 0 (I previous=I current), the current voltage (also referred to as present voltage) can be set as Vp for the amperometric process. However, if ΔI is greater than zero (Icurrent>Iprevious), at 44, the voltage can be continued to be increase linearly. The step in the linear voltage increase, 6V, can be constant or variable. The variable step can be set based on a predefined parameter (e.g., all steps need not be equal and, instead, may be based on ΔI—e.g., proportional to ΔI). By using ΔI in this manner (determining if negative or positive) to determine where and when the action ends, action 1—set initial voltage 26 can mimic step 1, right panel, of the traditional two-step method of FIG. 1 by automatically by self-detecting Vp.

After the Vp is detected and set as the initial condition, action 2—adjust voltage 28, as shown in FIG. 7, can be performed to ensure that the peak current is maintained by adjusting Vp to a present voltage when the current drops. During the amperometric process 24, the voltage value can be corrected so that a high current is still applied throughout the process. This is especially true during the electrodissolution process shown in FIG. 5, where the working electrode 20 dissolves.

At 48, a change in current (ΔI′) can be determined as Icurrent, the current measured at the present time, —I previous, the current measured at the previous time. At 50, when ΔI′<0 (indicating a current drop), the voltage increases from the present voltage value (initially Vp). This increase can be at a predefined step/rate. When there is no current drop, there is no change in the present voltage value and the present voltage is held at 58 (connection not shown in FIG. 7 for simplicity of illustration). When the change in current is between 0 and a predefined permissible current (Ix), the voltage can be held at Vp. Ix can be predefined according to the amperometric process or a property of the working electrode, based on a preselected value, or the like. After 50, if ΔI′<Ix, at 52, the voltage continues to increase until 0<ΔI′<Ix, where the voltage is held, at 58. At 54, if ΔI′<0, then the voltage is decreased from Vp and is held, at 58, when 0<ΔI′<Ix. However, if ΔI′>Ix after the voltage is decreased at 54, then the voltage is increased at 56 until 0<ΔI′<Ix, and the voltage is then held at 58.

Optionally, during or after action 2—adjust voltage 28, action 3—stop delivering voltage 30 to end the amperometric process can be performed, as shown in FIG. 8. At 60, a percent change in current drop (percent drop, ΔI %) can be determined by recording an instantaneous current (It) and taking a ratio of the instantaneous current It to a current at the start of a voltage hold (Io) and multiplying the ratio by 100% (ΔI %=(It/Io)×100%). At 62, if ΔI %<a threshold, the voltage delivery to the metal film working electrode 20 can be stopped (ending the amperometric process). However, at 64, if ΔI %>the threshold, the voltage delivery can be continued. The threshold can be a predefined percentage (e.g., based on the amperometric process, the electrochemical reaction, the working electrode, or other property of the process or system) that can be stored in the memory 14. As an example, the threshold can be any value between 1% and 50%.

### IV. Method

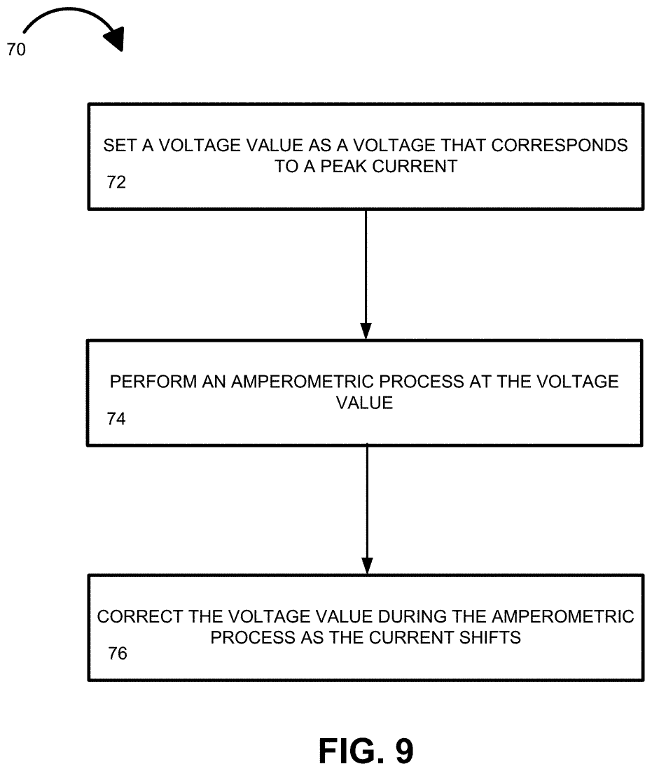

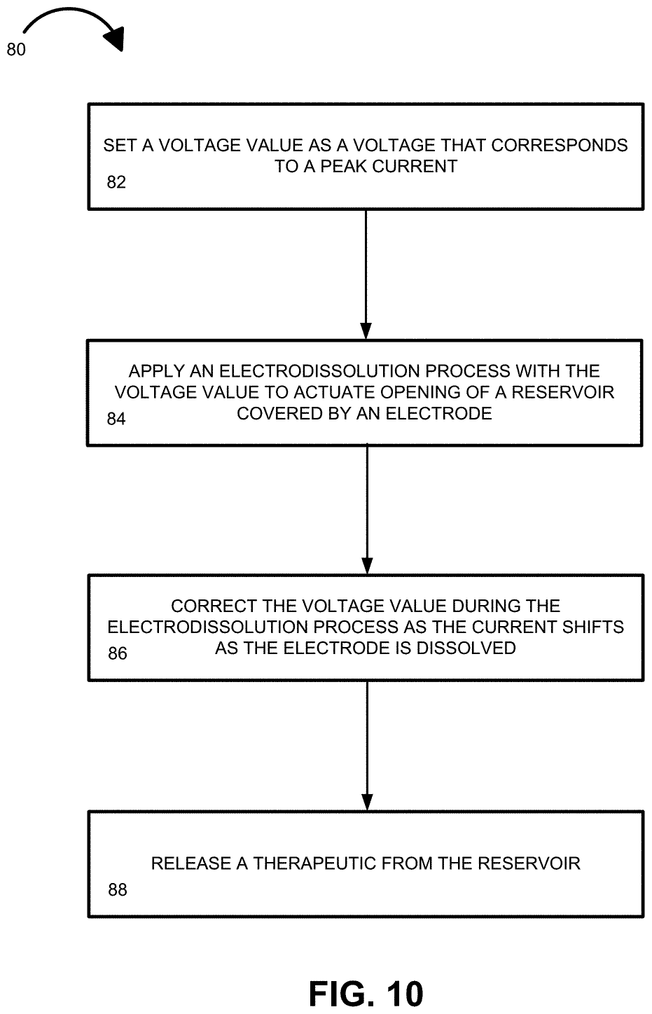

Another aspect of the present disclosure can include methods (FIGS. 9 and 10) that can achieve efficient electrochemical reactions within a hydrogel (e.g., silicone hydrogel) during an amperometric process using a two-electrode system by executing the linear sweep-hold method (shown in FIG. 2). It should be noted that the methods can be executed using a traditional three-electrode system and are not limited to a two-electrode system. The methods can be executed by the system 10 of FIG. 3, for example using the ophthalmic implementation of FIG. 4, using the control processes of FIGS. 5-8.

The methods 70 and 80 are illustrated as process flow diagrams with flowchart illustrations that can be implemented by one or more components of the system 10, as shown in FIG. 3. For purposes of simplicity, the methods 70 and 80 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 70 and 80.

FIG. 9 illustrates a method 70 for executing the one-step linear sweep-hold method to achieve an efficient amperometric process within a hydrogel (e.g., a silicone hydrogel). The amperometric process can be any process with a constant input voltage that facilitates one or more electrochemical reactions. For example, the amperometric process can facilitate electrodissolution.

At 72, a voltage value initial condition can be set (by controller 12) as a voltage that corresponds to a peak current (Vp). The voltage value (Vp) can be set according to action 1—set initial voltage 26, shown in FIG. 6. The Vp can be set and detected by: increasing a voltage linearly from a previous voltage value to a present voltage value; and determining a change in current between the previous voltage value and the present voltage value, such that when the change in current is greater than 0, increasing the voltage linearly from the present voltage value to another voltage value and determining another change in current; but when the change in current is less than 0, setting the Vp as the previous voltage value.

At 74, an amperometric process (e.g., amperometric process 24 using the two-electrode system of a working electrode 20 and reference/counter electrode 22) can be performed at the voltage value (using an electrical signal from generator 18 that is generated in response to one or more instructions by controller 12). At 76, the voltage value can be corrected (by controller 12 during the amperometric process as the current shifts. The voltage can be corrected according to action 2—adjust voltage 28, shown in FIG. 7. The voltage value can be corrected by: (1) determining a change in current between a present time and a previous time; and (2a) when the change in current is between 0 and a predefined permissible current (Ix), holding the voltage at Vp; (2b) when the change in current is greater than Ix, increasing the voltage until the change in current is between 0 and Ix and holding the voltage at the new increased voltage; or (2c) when the change in current is less than 0, decreasing the voltage and then increasing the voltage until the change in current is between 0 and Ix and holding the voltage at the new decreased voltage.

Optionally, the amperometric process can be ended by action 3—stop delivering voltage, as shown in FIG. 8. An instantaneous current (It) can be determined for each time point during the amperometric process. A current drop can be determined by taking a ratio of It and Io (the current at the start of a voltage hold). The percent change in current drop (% drop) can be determined by the ratio and multiplying by 100% (ΔI %=(It/Io)×100%). When the current drop percent exceeds a threshold, the voltage can be stopped to stop the amperometric process. As an example, the threshold can be any value between 1% and 50%.

FIG. 10 illustrates a method 80 for more efficient therapeutic release from a reservoir embedded within a hydrogel (e.g., a silicone hydrogel) using the one-step linear sweep-hold method to achieve an efficient amperometric process. In this example, the system of FIG. 4 can have the working electrode that doubles as the cover for the reservoir undergo electrodissolution. The electrodissolution can be of an electrode (e.g., working electrode 20) covering a reservoir 34 within a hydrogel of an ophthalmic device to facilitate controlled delivery of a therapeutic from the reservoir. Such an electrodissolution can employ a two-electrode system (e.g., working electrode 20 and reference/counter electrode 22), rather than a traditional three-electrode system (e.g., where the reference electrode and the counter electrode are two separate electrodes).

At 82, a voltage value can be set as a voltage that corresponds to a peak current (Vp). At 84, an electrodissolution process (shown in FIG. 4) can be applied with the electrical signal set to the voltage value to actuate opening of a reservoir (e.g., reservoir 34) covered by an electrode (e.g., working electrode 20, which may be a thin gold film). At 86, the voltage value can be corrected during the electrodissolution process as the current shifts as the electrode (e.g., working electrode 20) is dissolved. It should be noted that 82, 84, and 86 can use the actions 1, 2, and 3 shown in FIG. 5 and described in more detail in FIGS. 6-8. At 88, a therapeutic can be released from the reservoir (e.g., reservoir 34).

From the above description, those skilled in the art will perceive improvements, changes and modifications. Such improvements, changes and modifications are within the skill of one in the art and are intended to be covered by the appended claims. All patents, patent applications, and publications cited herein are incorporated by reference in their entirety.

What is claimed is:

1. A method comprising:
setting a voltage value to be a voltage that corresponds to a peak current (Vp); and
performing an amperometric process at the voltage value,
wherein the voltage value is corrected during the amperometric process as a current shifts.

2. The method of claim 1, further comprising detecting the Vp by:
increasing a voltage linearly from a previous voltage value to a present voltage value;
determining a change in current between the previous voltage value and the present voltage value;
when the change in current is greater than 0, increasing the voltage linearly from the present voltage value to another voltage value and determining another change in current; and
when the change in current is less than 0, setting the Vp as the previous voltage value.

3. The method of claim 1, further comprising correcting the voltage value by:
determining a change in current between a present time and a previous time;
when the change in current is between 0 and a predefined permissible current (Ix), holding the voltage at Vp;
when the change in current is greater than Ix, increasing the voltage until the change in current is between 0 and Ix and holding the voltage at the new increased voltage; and
when the change in current is less than 0, decreasing the voltage and then increasing the voltage until the change in current is between 0 and Ix and holding the voltage at the new decreased voltage.

4. The method of claim 3, further comprising recording an instantaneous current and determining a current drop percentage,
wherein when the current drop percentage exceeds a threshold, stopping the voltage.

5. The method of claim 4, wherein the threshold is from 1% to 50%.

6. The method of claim 1, wherein the amperometric process is performed using a two-electrode system.

7. The method of claim 1, wherein the amperometric process facilitates an electrochemical reaction.

8. A method comprising:
setting a voltage value to a voltage that corresponds to a peak current (Vp);
applying an electrodissolution process with the voltage set at Vp to actuate opening of a reservoir covered by an electrode, wherein the reservoir is positioned within a hydrogel of an ophthalmic device;
wherein the voltage value is corrected during the electrodissolution process as a current shifts as the electrode is dissolved; and
releasing a therapeutic from the reservoir.

9. The method of claim 8, wherein the electrodissolution process is applied using a two-electrode system.

10. The method of claim 9, wherein the electrode is a working electrode of a two-electrode system.

11. The method of claim 8, wherein the electrode is a metal film holding the drug within the reservoir.

12. The method of claim 8, wherein the voltage value is set by self-detecting a voltage that corresponds to the peak current (Vp) by:
increasing a voltage linearly from a previous voltage value to a present voltage value;
determining a change in current between the previous voltage value and the present voltage value;
when the change in current is greater than 0, increasing the voltage linearly from the present voltage value to another voltage value and determining another change in current; and
when the change in current is less than 0, setting the Vp as the previous voltage value.

13. The method of claim 12, further comprising adjusting the voltage value during the electrodissolution process to maintain the peak current.

14. The method of claim 13, wherein the voltage value is adjusted by:
determining a change in current between a present time and a previous time;
when the change in current is between 0 and a predefined permissible current (Ix), holding the voltage at Vp;
when the change in current is greater than Ix, increasing the voltage until the change in current is between 0 and Ix and holding the voltage value at the new increased voltage; and
when the change in current is less than 0, decreasing the voltage and then increasing the voltage until the change in current is between 0 and Ix and holding the voltage value at the new decreased voltage.

15. The method of claim 14, further comprising recording an instantaneous current and determining a current drop percentage,
wherein when the current drop percentage exceeds a threshold indicating that the electrode has been dissolved enough to release the drug, stopping application of the voltage.

16. The method of claim 15, wherein the threshold is from 1% to 50%.

17. A system comprising:
a controller comprising a memory storing instructions and a processor configured to access the memory and execute the instructions to set a voltage value that corresponds to a peak current (Vp); and
a generator to generate an electrical signal at the voltage value and send the electrical signal to an electrode to perform an amperometric process at a peak current,
wherein the controller is configured to correct the voltage value as the peak current shifts during the amperometric process.

18. The system of claim 17, wherein the generator is coupled to a two-electrode system, wherein the two-electrode system comprises a working electrode and a counter/reference electrode within a hydrogel ophthalmic device.

19. The system of claim 18, wherein the working electrode comprises a metal film covering a reservoir and configured for electrodissolution by the amperometric process.

20. The system of claim 19, wherein the metal film comprises gold.
